(19) United States
(12) Patent Application Publication
(10) Pub. No.: US 2022/0296375 A1
(43) Pub. Date: Sep. 22, 2022

(54) CRANIAL IMPLANT DEVICES AND RELATED METHODS FOR MONITORING BIOMETRIC DATA

(71) Applicant: THE JOHNS HOPKINS UNIVERSITY, Baltimore, MD (US)

(72) Inventors: Chad GORDON, Cockeysville, MD (US); Netanel BEN-SHALOM, Baltimore, MD (US)

(21) Appl. No.: 17/642,284

(22) PCT Filed: Sep. 11, 2020
(86) PCT No.: PCT/US2020/050416

§ 371 Date: Mar. 11, 2022

Related U.S. Application Data
(60) Provisional application No. 62/899,976, filed on Sep. 13, 2019.

(51) Int. Cl. A61F 2/28; A61B 5/03; A61B 5/00; G01S 15/89

(52) U.S. Cl. CPC A61F 2/2875; A61B 5/031; A61B 5/6864; G01S 15/8906; A61F 2002/3009

(57) ABSTRACT

Provided herein are cranial implant devices that include a cranial implant housing configured for subgaleal scalp implantation within, beneath, and/or over at least one cranial opening of a subject. The cranial implant housing comprises a substantially anatomically-compatible shape and is fabricated from one or more sonolucent materials that permit transmission of mechanical waves through the sonolucent materials when the cranial implant device is subgaleally implanted. The cranial implant housing also includes a pressure sensor operably connected to the cranial implant housing, which pressure sensor is configured to sense intracranial pressure (ICR). The cranial implant housing also includes at least a first controller operably connected to the pressure sensor, which first controller is configured to selectively effect the pressure sensor to sense the ICR within a cranium of the subject to generate ICP data and to transmit the ICP data to an ICP data receiver. In addition, the cranial implant housing also includes a power source operably connected or connectable at least to the first controller. Other aspects are directed to various related systems, computer readable media, and methods.

### CROSS REFERENCE TO RELATED APPLICATION

This application claims the benefit of U.S. Provisional Application No. 62/899,976 filed Sep. 13, 2019, the disclosure of which is hereby incorporated by reference in its entirety.

### FIELD

The present disclosure relates generally to cranial implants and, more particularly, to cranial implants that provide a wide array of diagnostic, therapeutic and/or monitoring capabilities.

### BACKGROUND

In the field of neurosurgery, neuromonitoring and brain visualization plays a robust role for diagnosis, monitoring, and surveillance of acute brain injury and other brain-related conditions or pathologies. Current methods, such as computed tomography (CT) and magnetic resonance imaging (MRI) are not always available, expensive, and do not provide continuous, real-time monitoring capabilities. Other neuromonitoring techniques, such as many pre-existing cabled intracranial pressure (ICP) monitors carry a relatively high risk of infection, which typically limits their use to at most five days of continuous implantation. This creates a significant problem, since conditions, such as intracranial hypertension (intracranial-HTN) frequently exhibit a delayed onset with second peaks often starting on days 9-11 after, for example, an acute brain injury is initially sustained.

Accordingly, there remains a need for approaches that enable continuous, real-time neuromonitoring of various types of biometric data over extended or indefinite periods of time.

### SUMMARY

This application discloses various cranial implant devices that are configured for subgaleal scalp implantation within, beneath, and/or over cranial openings in subjects for performing a wide array of diagnostic, therapeutic and/or monitoring applications. The devices are typically fabricated from one or more sonolucent or other materials that permit transmission of one or more mechanical waves and/or electromagnetic waves through the sonolucent or other materials, even upon subgaleal implantation in subjects to permit the acquisition of ultrasound and other biometric data. The cranial implant devices disclosed herein also include pressure sensors that are operable to sense intracranial pressure (ICP) while implanted in subjects. In addition, the cranial implant devices are generally configured to transmit ICP and/or other biometric data to data receivers continuously and in substantially real-time so healthcare providers can monitor courses of treatment, among other applications. Further, once implanted in subjects, the devices may remain in place for indefinite durations with minimal risk of infection. The devices also have substantially anatomically-compatible shapes such that they are essentially non-detectable upon implantation in subjects. In addition to cranial implant devices, related systems and methods are also provided.

In one aspect, this disclosure provides a cranial implant device that includes at least one cranial implant housing configured for subgaleal scalp implantation within, beneath, and/or over at least one cranial opening of a subject. The cranial implant housing comprises a substantially anatomically-compatible shape and is fabricated from one or more sonolucent materials that permit transmission of one or more mechanical waves (e.g., ultrasound waves or the like) through the sonolucent materials when the cranial implant device is subgaleally implanted within, beneath, and/or over the cranial opening of the subject. The cranial implant device also includes at least one pressure sensor (e.g., a microsensor or the like) operably connected to the cranial implant housing, which pressure sensor is configured to sense intracranial pressure (ICP). The cranial implant device additionally includes at least a first controller operably connected to the pressure sensor. The controller is configured to selectively effect the pressure sensor to sense the ICP within a cranium of the subject to generate ICP data and to transmit the ICP data to at least one ICP data receiver when the cranial implant device is subgalealy implanted within, beneath, and/or over the cranial opening of the subject. In addition, the cranial implant device also includes at least one power source operably connected or connectable at least to the first controller. In some embodiments, the cranial implant device comprises a standardized form, whereas in other exemplary embodiments, the cranial implant device comprises a form that is customized for the subject. In certain embodiments, a kit comprises the cranial implant device.

In some embodiments, the cranial implant device is structured for subgaleal scalp implantation within, beneath, and/or over at least one burr hole in a skull or in a skull flap of the subject. In certain embodiments, an autologous skull flap comprises at least a portion of the cranial implant device. In some embodiments, an alloplastic cranial implant comprises at least a portion of the cranial implant device.

In certain embodiments, the cranial implant housing comprises one or more attachment features configured to at least partially effect attachment of the cranial implant device to the cranium of the subject when the cranial implant device is subgalealy implanted within, beneath, and/or over the cranial opening of the subject. In some embodiments, the pressure sensor, the first controller, and/or the power source are at least partially embedded in the cranial implant housing. In certain embodiments, the power source is external to the cranial implant housing. In some embodiments, the cranial implant housing comprises polymethylmethacrylate (PMMA), room-temperature-vulcanizing (RTV) silicone, polydimethylsiloxane (PDMS), epoxy, polyetheretherketone (PEEK), and/or metamaterials. In certain embodiments, the pressure sensor extends from at least one surface of the cranial implant housing into the cranium of the subject when the cranial implant device is subgalealy implanted within, beneath, and/or over the cranial opening of the subject. In some embodiments, the cranial implant device further comprises at least one self-sealing access port disposed through the cranial implant housing, which self-sealing access port permits cerebrospinal fluid (CSF) to be aspirated from the subject when the cranial implant device is subgalealy implanted within, beneath, and/or over the cranial opening of the subject.

In certain embodiments, the first controller is operably connected to the ICP data receiver via at least one wired connection and wherein the first controller is further configured to transmit the ICP data to the ICP data receiver via the wired connection when the cranial implant device is subgalealy implanted within, beneath, and/or over the cranial opening of the subject. In some of these embodiments, the first controller is configured to continuously transmit the ICP data to the ICP data receiver. In certain of these embodiments, the first controller is configured to transmit the ICP data in substantially real-time to the ICP data receiver. In some embodiments, the first controller is further configured for wireless connectivity to the ICP data receiver and to wirelessly transmit the ICP data to the ICP data receiver when the cranial implant device is subgalealy implanted within, beneath, and/or over the cranial opening of the subject. In some of these embodiments, the first controller is configured to continuously transmit the ICP data to the ICP data receiver. In certain of these embodiments, the first controller is configured to transmit the ICP data in substantially real-time to the ICP data receiver. In some of these embodiments, the ICP data receiver comprises a mobile device selected from the group consisting of: a telephone, a tablet computer, and a notebook computer.

In some embodiments, the cranial implant device further comprises one or more additional sensors components operably connected to the cranial implant housing, the first controller, and/or the power source, which additional sensors components are configured to sense additional biometric data from within the cranium of the subject when the cranial implant device is subgalealy implanted within, beneath, and/or over the cranial opening of the subject. In some of these embodiments, the additional biometric data comprises brain temperature, lactate level, oxygen level, and/or carbon dioxide level.

In another aspect, the present disclosure presents a cranial implant device that includes at least one acoustic, optical, and/or photoacoustic lens element comprising one or more electromagnetically translucent, electromagnetically transparent, sonolucent, and/or acoustically active materials, and at least one biometric data sensor operably connected to the acoustic, optical, and/or photoacoustic lens element. The biometric data sensor is configured to sense biometric data from a cranium of a subject and to transmit the biometric data to at least one ICP data receiver. The cranial implant device is also structured for subgaleal scalp implantation within, beneath, and/or over at least one cranial opening of the subject. The cranial implant device additionally comprises a substantially anatomically-compatible shape. In addition, the cranial implant device further permits transcranial therapeutic ultrasound, transcranial diagnostic ultrasound, photoacoustic imaging, electromagnetic wave diagnostic imaging, and/or electromagnetic wave therapeutic intervention of intracranial matter of the subject via the acoustic, optical, and/or photoacoustic lens element when the cranial implant device is subgalealy implanted within, beneath, and/or over the at least one cranial opening of the subject. In some of these embodiments, a focal point of the acoustic, optical, and/or photoacoustic lens element is adjustable. In certain of these embodiments, the biometric data sensor is selected from the group consisting of: an intracranial pressure sensor, a temperature sensor, a lactate sensor, an oxygen sensor, and a carbon dioxide sensor.

In another aspect, the present disclosure relates to an implant device that includes at least one implant housing configured for subgaleal scalp implantation within, beneath, and/or over at least one bodily opening of a subject. The implant housing comprises a substantially anatomically-compatible shape and is fabricated from one or more sonolucent materials that permit transmission of one or more mechanical waves through the sonolucent materials when the implant device is subgalealy implanted within, beneath, and/or over the bodily opening of the subject. The implant device also includes at least one pressure sensor operably connected to the implant housing, which pressure sensor is configured to sense intrabodily pressure (IBP). The implant device also includes at least a first controller operably connected to the pressure sensor. The first controller is configured to selectively effect the pressure sensor to sense the IBP within a bodily cavity and/or organ of the subject to generate IBP data and to transmit the IBP data to at least one IBP data receiver when the implant device is subgalealy implanted within, beneath, and/or over the bodily opening of the subject. In addition, the implant device also includes at least one power source operably connected or connectable at least to the first controller. In some of these embodiments, a rib cage of the subject comprises bodily opening. In certain embodiments, the bodily cavity and/or organ comprises a liver, a lung, and/or a heart of the subject.

In some embodiments, the first controller is operably connected to the IBP data receiver via at least one wired connection and wherein the first controller is further configured to transmit the IBP data to the IBP data receiver via the wired connection when the cranial implant device is subgalealy implanted within, beneath, and/or over the cranial opening of the subject. In certain of these embodiments, the first controller is configured to continuously transmit the IBP data to the IBP data receiver. In some of these embodiments, the first controller is configured to transmit the IBP data in substantially real-time to the IBP data receiver. In certain embodiments, the first controller is further configured for wireless connectivity to the IBP data receiver and to wirelessly transmit the IBP data to the IBP data receiver when the cranial implant device is subgalealy implanted within, beneath, and/or over the cranial opening of the subject. In certain of these embodiments, the first controller is configured to continuously transmit the IBP data to the IBP receiver. In some of these embodiments, the first controller is configured to transmit the IBP data in substantially real-time to the IBP data receiver. In certain of these embodiments, the IBP data receiver comprises a mobile device selected from the group consisting of: a telephone, a tablet computer, and a notebook computer.

In another aspect, the present disclosure presents a system that includes at least one cranial implant device. The cranial implant device includes at least one cranial implant housing configured for subgaleal scalp implantation within, beneath, and/or over at least one cranial opening of a subject. The cranial implant housing comprises a substantially anatomically-compatible shape and is fabricated from one or more sonolucent materials that permit transmission of one or more mechanical waves through the sonolucent materials when the cranial implant device is subgalealy implanted within, beneath, and/or over the cranial opening of the subject. The cranial implant device also includes at least one pressure sensor operably connected to the cranial implant housing, which pressure sensor is configured to sense intracranial pressure (ICP). The cranial implant device also includes at least a first controller operably connected to the pressure sensor. The first controller is configured to selectively effect the pressure sensor to sense the ICP within a cranium of the subject to generate ICP data and to transmit the ICP data to at least one ICP data receiver when the cranial implant device is subgalealy implanted within, beneath, and/or over the cranial opening of the subject. In addition, the cranial implant device also includes at least one power source operably connected or connectable at least to the first controller. The system also includes at least one transmission and/or receiver device configured to transmit and/or receive the mechanical waves through the sonolucent materials when the cranial implant device is subgalealy implanted within, beneath, and/or over the cranial opening of the subject. In addition, the system also includes at least a second controller operably connected to the transmission and/or receiver device, which second controller comprises, or is capable of accessing, computer readable media comprising non-transitory computer-executable instructions which, when executed by at least one electronic processor cause the transmission and/or receiver device to transmit and/or receive the mechanical waves through the sonolucent materials of the cranial implant device when the cranial implant device is subgalealy implanted within, beneath, and/or over the cranial opening of the subject and when the transmission and/or receiver device is positioned in communication with the cranial implant device. In some embodiments, the cranial implant device comprises a standardized form, whereas in other embodiments, the cranial implant device comprises a form that is customized for the subject.

In some embodiments of the systems disclosed herein, the cranial implant device is structured for subgaleal scalp implantation within, beneath, and/or over at least one burr hole in a skull or in a skull flap of the subject. In certain embodiments, an autologous skull flap comprises at least a portion of the cranial implant device. In some embodiments, an alloplastic cranial implant comprises at least a portion of the cranial implant device.

In certain embodiments, the systems disclosed herein further comprise at least one adjustable or fixed external lens element configured to further focus the mechanical waves transmitted through the sonolucent material when the adjustable or fixed external lens element is positioned in communication with the cranial implant device and the transmission and/or receiver device. In some embodiments, the transmission and/or receiver device comprises at least one sensing mechanism configured to store, analyze, and/or modify echo signals transmitted through the sonolucent material in a time domain. In certain embodiments, the transmission and/or receiver device comprises at least one sensing mechanism configured to store, analyze, and/or modify echo signals transmitted through the sonolucent material in a frequency domain.

In some embodiments of the systems disclosed herein, the first controller is operably connected to the ICP data receiver via at least one wired connection and wherein the first controller is configured to transmit the ICP data to the ICP data receiver via the wired connection when the cranial implant device is subgalealy implanted within, beneath, and/or over the cranial opening of the subject. In certain embodiments, the first controller is configured for wireless connectivity to the ICP data receiver and to wirelessly transmit the ICP data to the ICP data receiver when the cranial implant device is subgalealy implanted within, beneath, and/or over the cranial opening of the subject. In some embodiments, the ICP data receiver comprises at least one antenna. In certain embodiments, the mechanical waves comprise ultrasound waves and wherein the second controller is configured to process the ultrasound waves received through the sonolucent materials of the cranial implant device to generate one or more ultrasound images. In some embodiments, the transmission and/or receiver device comprises at least one ultrasound transducer that is configured to send and receive ultrasound waves transmitted through the sonolucent materials. In certain of these embodiments, the ultrasound transducer comprises at least one cross-sectional shape that comprises at least one concave, convex, and/or flat portion. In some embodiments, the non-transitory computer-executable instructions which, when executed by the at least one electronic processor, cause the at least one ultrasound transducer to implement an imaging sequence and/or an imaging technique. In some embodiments, the imaging sequence and/or the imaging technique comprises one or more selectable parameters of the at least one ultrasound transducer that are selected from the group consisting of: a number of elements, a center frequency, a speed of sound, a wave length, an array pitch, a sampling frequency, and an emission pulse. In certain embodiments, the imaging sequence and/or the imaging technique comprises reassembling and/or normalizing ultrasound images transmitted through the sonolucent materials in substantially real-time.

In another aspect, the present disclosure presents a computer readable media comprising non-transitory computer-executable instructions which, when executed by at least one electronic processor perform at least: sensing intracranial pressure (ICP) within a cranium of a subject to generate ICP data using at least one pressure sensor of at least one cranial implant device subgalealy implanted within, beneath, and/or over at least one cranial opening of the subject. The cranial implant device comprises at least one cranial implant housing comprising a substantially anatomically-compatible shape and is fabricated from one or more sonolucent materials that permit transmission of one or more mechanical waves through the sonolucent materials. The cranial implant device also comprises the pressure sensor operably connected to the cranial implant housing. The cranial implant device also comprises at least a first controller operably connected to the pressure sensor. The first controller is configured to selectively effect the pressure sensor to sense the ICP within the cranium of the subject to generate the ICP data and to transmit the ICP data to at least one ICP data receiver. In addition, the cranial implant device also comprises at least one power source operably connected or connectable at least to the first controller. The non-transitory computer-executable instructions which, when executed by at least one electronic processor perform at least: transmitting the ICP data to the ICP data receiver from the first controller.

In another aspect, the present disclosure presents a method of obtaining diagnostic information from, and/or administering therapy to, a subject. The method includes implanting at least one cranial implant device subgalealy within, beneath, and/or over at least one cranial opening of the subject. The cranial implant device includes at least one cranial implant housing comprising a substantially anatomically-compatible shape and is fabricated from one or more sonolucent materials that permit transmission of one or more mechanical waves through the sonolucent materials. The cranial implant device also includes at least one pressure sensor operably connected to the cranial implant housing, which pressure sensor is configured to sense intracranial pressure (ICP). The cranial implant device also includes at least a first controller operably connected to the pressure sensor. The first controller is configured to selectively effect the pressure sensor to sense the ICP within a cranium of the subject to generate ICP data and to transmit the ICP data to at least one ICP data receiver. In addition, the cranial implant device also includes at least one power source operably connected or connectable at least to the first controller. The method also includes sensing the ICP data using the cranial implant device, and transmitting the ICP data to the ICP data receiver from the first controller. In addition, the method also includes transmitting and/or receiving the mechanical waves through the sonolucent materials of the cranial implant device into and/or from intracranial matter of the subject using at least one transmission and/or receiver device, thereby obtaining the diagnostic information from, and/or administering the therapy to, the subject. In some embodiments, the cranial implant device comprises a standardized form, whereas in other embodiments, the cranial implant device comprises a form that is customized for the subject.

In some embodiments, the method comprises implanting the cranial implant device subgalealy within, beneath, and/or over at least one burr hole in a skull of the subject. In certain embodiments, the method includes affixing the cranial implant device to a skull of the subject using one or more screws and/or one or more chemical bonding agents. In certain embodiments, the method includes correlating the ICP data with the transmitted and/or received mechanical waves to effect a synergistic diagnosis of, and/or therapeutic administration to, the subject. In some embodiments, the mechanical waves comprise ultrasound waves and wherein the transmission and/or receiver device processes the ultrasound waves received through the sonolucent materials of the cranial implant device to generate one or more ultrasound images.

In certain embodiments, the first controller is operably connected to the ICP data receiver via at least one wired connection and wherein the method comprises transmitting the ICP data to the ICP data receiver via the wired connection. In some embodiments, the first controller is configured for wireless connectivity to the ICP data receiver and wherein the method comprises wirelessly transmitting the ICP data to the ICP data receiver. In certain embodiments, the method includes continuously transmitting the ICP data to the ICP data receiver from the first controller. In some embodiments, the method includes transmitting the ICP data to the ICP data receiver from the first controller in substantially real-time. In certain embodiments, the method includes retaining the cranial implant device subgalealy implanted within, beneath, and/or over the cranial opening of the subject for an indefinite duration.

In certain embodiments, the method includes implanting multiple cranial implant devices subgalealy within, beneath, and/or over multiple cranial openings of the subject. In some embodiments, the method includes implanting the cranial implant device subgalealy within, beneath, and/or over the cranial opening of the subject while performing at least one neurosurgical procedure on the subject selected from the group consisting of: an aneurysm surgery, a brain tumor removal surgery, a hydrocephalus surgery, a brain neurodegenerative disease surgery, a carotid bypass surgery, a decompression craniectomy for head trauma, reconstructive cranioplasty, standard fashion craniotomy, an epilepsy surgery, and the like. In certain embodiments, the subject has one or more neurological diseases or conditions selected from the group consisting of: an aneurysm, a brain tumor, hydrocephalus, a brain neurodegenerative disease, epilepsy, subarachnoid-related cerebral vasospasm, Moyamoya disease, a cerebral artery blockage, meningitis, encephalitis, a brain abscess, a cerebral edema, traumatic brain injury and pseurotumor cerebri. In some embodiments, the subject sustained a brain injury. Typically, the subject is a mammal (e.g., a human).

In another aspect, the present disclosure presents a surgical method, the method comprising surgically implanting at least one cranial implant device within, beneath, and/or over at least one cranial opening of a subject. The cranial implant device comprises at least one cranial implant housing configured for subgaleal scalp implantation within, beneath, and/or over the cranial opening of the subject. The cranial implant housing comprises a substantially anatomically-compatible shape and is fabricated from one or more sonolucent materials that permit transmission of one or more mechanical waves through the sonolucent materials when the cranial implant device is subgalealy implanted within, beneath, and/or over the cranial opening of the subject. The cranial implant device also comprises at least one pressure sensor operably connected to the cranial implant housing, which pressure sensor is configured to sense intracranial pressure (ICP). The cranial implant device also comprises at least a first controller operably connected to the pressure sensor. The first controller is configured to selectively effect the pressure sensor to sense the ICP within a cranium of the subject to generate ICP data and to transmit the ICP data to at least one the ICP data receiver when the cranial implant device is subgalealy implanted within, beneath, and/or over the cranial opening of the subject. In addition, the cranial implant device also comprises at least one power source operably connected or connectable at least to the first controller. In some embodiments, the sonolucent materials form at least one lens element. In some of these embodiments, a focal point of the lens element is adjustable and wherein the method further comprises adjusting the focal point of the lens element. In some embodiments, the method further comprises ablating tissue within the cranium of the subject using the lens element.

In another aspect, the present disclosure presents a method of fabricating a cranial implant device. The method includes forming at least one cranial implant housing from at least one or more sonolucent materials such that the cranial implant housing comprises a substantially anatomically-compatible shape. The method also includes positioning at least one pressure sensor in operable connection with the cranial implant housing, which pressure sensor is configured to sense intracranial pressure (ICP). The method also includes positioning at least a first controller in operable connection with the pressure sensor. The first controller is configured to selectively effect the pressure sensor to sense the ICP within a cranium of a subject to generate ICP data and to transmit the ICP data to at least one ICP data receiver when the cranial implant device is subgalealy implanted within, beneath, and/or over a cranial opening of the subject. In addition, the method also includes positioning at least one power source in operable connection at least with the first controller, thereby fabricating the cranial implant device.

The sonolucent materials typically form, and/or comprise, at least one lens element. The lens elements of in the cranial implant devices disclosed herein include various embodiments. In some embodiments, a focal point of the lens element is adjustable. In certain embodiments, the lens element is interchangeable with another lens element. In some embodiments, the cranial implant device comprises 2, 3, 4, 5, 6, 7, 8, 9, 10 or more lens elements. In certain embodiments, the lens element comprises a three-dimensional structure configured to reduce a speed of sound transmitted through the lens element. In some embodiments, the lens element comprises one or more wave-guides. In certain embodiments, the lens element comprises one or more acoustic metamaterials and/or one or more phononic crystals. In some embodiments, the lens element comprises at least one metamaterial having a negative refractive index and at least one other material having a subwavelength microstructure. In certain embodiments, the lens element comprises a plano-convex lens, a biconvex lens, a plano-concave lens, a biconcave lens, a positive meniscus lens, a negative meniscus lens, a converging Fresnel lens, and/or a diverging Fresnel lens. In some embodiments, the lens element comprises a curved or rectilinear cross-sectional shape. In some embodiments, the lens element comprises at least one material that is modified to increase or decrease a speed of sound transmitted through the material.

In some embodiments, the lens element comprises at least one substantially flat diverging lens comprising at least two different materials, wherein at least a first material transmits sound at a higher speed than a tissue of the subject, and wherein at least a second material transmits sound at a lower speed than the tissue of the subject. In certain embodiments, the lens element comprises at least one diverging compound concave lens comprising at least two different materials, wherein at least a first material transmits sound at a higher speed than at least a second material, and wherein the second material is positioned closer to a scalp of the subject than the first material when the cranial implant device is subgalealy implanted within, beneath, and/or over the at least one cranial opening of the subject. In some embodiments, the lens element comprises at least one diverging compound convex lens comprising at least two different materials, wherein at least a first material transmits sound at a lower speed than at least a second material, and wherein the second material is positioned closer to a scalp of the subject than the first material when the cranial implant device is subgalealy implanted within, beneath, and/or over the at least one cranial opening of the subject.

In certain embodiments, the lens element comprises at least two lenses, wherein at least a first lens comprises a different ratio of focal distance to lens diameter than at least a second lens. In some embodiments, the lens element comprises at least one diverging lens that transmits sound at a lower speed than a tissue of the subject. In certain embodiments, the lens element comprises at least one material configured to receive optic beams reflected off the intracranial matter of the subject and emit ultrasonic waves in response when the cranial implant device is subgalealy implanted within, beneath, and/or over the at least one cranial opening of the subject. In some embodiments, the lens element comprises at least one diverging lens that transmits sound at a lower speed than a tissue of the subject. In certain embodiments, the lens element comprises at least one diverging lens that transmits sound at a higher speed than a tissue of the subject.

### BRIEF DESCRIPTION OF THE DRAWINGS

The accompanying drawings, which are incorporated in and constitute a part of this specification, illustrate certain embodiments, and together with the written description, serve to explain certain principles of the cranial implant devices, kits, systems, and related methods disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A schematically shows a method of implanting a left-sided, full-thickness skull resection (outlined by a cut region) into the resected portion of a skull from a perspective view according to one exemplary embodiment.

FIG. 1B schematically shows the resulting implantation of the skull flap into the resected portion of the skull of FIG. 1A along with cranial implant devices into burr holes or portions thereof in the skull flap and skull.

FIG. 2A schematically shows a cranial implant device from a top view according to one exemplary embodiment.

FIG. 2B schematically shows the cranial implant device of FIG. 2A from a side view.

FIG. 2C schematically shows the cranial implant device of FIG. 2A positioned within, beneath, and over a cranial opening of a subject from a sectional side view.

FIG. 3A schematically depicts an implanted cranial implant device comprising a biconvex lens element from a sectional side view according to one exemplary embodiment.

FIG. 3B schematically depicts an implanted cranial implant device comprising a biconvex lens element with other material disposed on both sides of the lens element from a sectional side view according to one exemplary embodiment.

FIG. 3C schematically depicts an implanted cranial implant device comprising a biconcave lens element with other material disposed on both sides of the lens element from a sectional side view according to one exemplary embodiment.

FIG. 3D schematically depicts an implanted cranial implant device comprising two biconvex lens elements with other layers of materials disposed between the lens element from a sectional side view according to one exemplary embodiment.

FIG. 3E schematically depicts an implanted cranial implant device comprising a convex lens element with other layers of material disposed on both sides of the lens element from a sectional side view according to one exemplary embodiment.

FIG. 3F schematically depicts an implanted cranial implant device comprising a convex lens element with other layers of material disposed on both sides of the lens element from a sectional side view according to one exemplary embodiment.

FIG. 3G schematically depicts an implanted cranial implant device comprising a Fresnel lens element with other layers of material disposed on both sides of the lens element from a sectional side view according to one exemplary embodiment.

FIG. 3H schematically depicts an implanted cranial implant device comprising multiple layers of different materials from a sectional side view according to one exemplary embodiment.

FIG. 3I schematically depicts an implanted cranial implant device comprising a biconvex lens element with other layers of material disposed on one side of the lens element from a sectional side view according to one exemplary embodiment.

FIG. 3J schematically depicts the implanted cranial implant device from FIG. 3B with an external lens element positioned in communication with the implanted cranial implant device and a transmission and/or receiver device from a sectional side view according to one exemplary embodiment.

FIG. 4 is a flow chart that schematically depicts exemplary method steps of obtaining diagnostic information from, and/or administering therapy to, a subject according to one exemplary embodiment.

FIG. 5 is a flow chart that schematically depicts exemplary method steps of fabricating a cranial implant device according to one exemplary embodiment.

FIG. 6 schematically shows a system according to one exemplary embodiment.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, cranial implant devices, and component parts, the following terminology, and grammatical variants thereof, will be used in accordance with the definitions set forth below.

About: As used herein, “about,” “approximately,” or “substantially” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Acoustic Lens: As used herein, “acoustic lens” refers to a configuration of one or more materials that allow the transmission of mechanical waves (e.g., sound) through those materials. In some configurations, those materials also spread and/or converge mechanical waves (e.g., sound) that are transmitted through those materials.

Alloplastic: As used herein, “alloplastic” in the context of cranial implants refers to a cranial implant that does not include material obtained or otherwise derived from a given subject into whom that implant is implanted. In some applications, alloplastic cranial implants comprise materials, such as medical grade metals (e.g., titanium, stainless steel, or the like), plastics, and non-autologous biological materials.

Autologous: As used herein, “autologous” in the context of cranial implants refers to a cranial implant that includes biological material (e.g., a skull bone flap, transplanted biological matter, etc.) obtained or otherwise derived from a given subject into whom that implant is implanted.

Burr Hole: As used herein, “burr hole” refers to a cranial opening or hole intentionally created by a healthcare provider through the skull of a subject as part of a given medical intervention. Burr-holes can have a range of diameters from about 1 mm to about 20 mm or larger (e.g., 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, and 19 mm). A standard burr-hole diameter is typically about 14 mm. The term “burr hole” is sometimes used interchangeably with the terms “keyhole” or “MacCarty keyhole.”

Communication: As used herein, “communication” in the context of transmission and/or receiver devices and cranial implant devices refers to a positioning or proximity of those devices relative to one another such that the transmission and/or receiver devices is able to transmit and/or receive mechanical and/or electromagnetic waves through the cranial implant devices.

Customized: As used herein, “customized” in the context of cranial implant shapes refers to a shape that has been created at the point of fabrication specifically for an individual subject. In some embodiments, for example, custom craniofacial implants (CCIs) are designed and manufactured using computer-aided design/manufacturing (CAD/CAM) based in part on fine cut preoperative computed tomography (CT) scans and three-dimensional reconstruction (+/− stereolithographic models).

Electromagnetic Wave: As used herein, “electromagnetic wave” refers to a wave of the electromagnetic spectrum that propagates through space and carries electromagnetic radiant energy.

Mechanical Wave: As used herein, “mechanical wave” refers to a wave that is an oscillation of matter, and thus transfers energy through a medium.

Metamaterial: As used herein, “metamaterial” refers to a synthetic material having a structure engineered to exhibit one or more properties (e.g., a negative refractive index, etc.) not typically observed in naturally occurring materials.

Optical Lens: As used herein, “optical lens” refers to a configuration of one or more materials that allow the transmission of electromagnetic waves (e.g., light) through those materials. In some configurations, those materials also spread and/or converge electromagnetic waves (e.g., light) that are transmitted through those materials.

Photoacoustic Lens: As used herein, “photoacoustic lens” refers to a configuration of one or more materials that allow the transmission of mechanical waves (e.g., sound) and electromagnetic waves (e.g., light) through those materials. In some configurations, those materials also spread and/or converge mechanical waves (e.g., sound) and/or electromagnetic waves (e.g., light) that are transmitted through those materials. In certain applications, photoacoustic lenses are used for photoacoustic or optoacoustic imaging, which is a biomedical imaging technique based on the photoacoustic or optoacoustic effect in which mechanical waves are formed following the absorption of electromagnetic waves (e.g., laser light, gamma radiation, X-rays, microwaves, radio frequency waves, etc.) in a given material, such as intracranial matter or other biological tissue.

Sonolucent Material: As used herein, “sonolucent material” refers a material that permits the transmission of mechanical waves (e.g., ultrasonic waves) through the material substantially without producing echoes or other distortions (e.g., caused by the reflection of those mechanical waves).

Standardized: As used herein, “standardized” in the context of cranial implant shapes refers to a shape that has not been created at the point of fabrication specifically for any individual subject. Instead, a standardized implant shape is typically selected for ease of readily reproducible manufacture. Cranial implants having standardized shapes may also be referred to as “off the shelf” neurological implants.

Subgaleal: As used herein, “subgaleal” refers to an anatomical location substantially below the galea aponeurotica of a given subject.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, an individual that has sustained or is suspected of having sustained a brain injury, or an individual that is in need of therapy or suspected of needing therapy. The terms “individual” or “patient” are intended to be interchangeable with “subject.” For example, a subject can be an individual who has been diagnosed with having a cancer, is going to receive a cancer therapy, and/or has received at least one cancer therapy. The subject can be in remission of a cancer.

Substantially Anatomically-Compatible Shape: As used herein, “substantially anatomically-compatible shape” in the context of cranial implant devices refers to a shape such that when the device is implanted in a subject, the device is essentially visually imperceptible in the absence of, for example, analytical imaging, such as X-ray-based imaging or the like.

Translucent: As used herein, “translucent” or “semitransparent” refers to a property of a material that allows the transmission and diffusion of electromagnetic waves through the material, such that objects or matter lying beyond the material are not seen with substantial clarity.

Transparent: As used herein, “transparent” refers to a property of a material that permits the transmission of electromagnetic waves through the material without appreciable scattering, such that objects or matter lying beyond the material are seen with substantial clarity.

### DETAILED DESCRIPTION

This application discloses various cranial implant devices that are used to monitor and in certain embodiments, wirelessly transmit substantially real-time intracranial pressure (ICP) data to healthcare providers. The cranial implant devices are typically made of sonolucent or other materials that create an acoustic window for ultrasound imaging of the brain in substantially real-time, among other applications. The cranial implant devices disclosed herein also have embodiments with wired configurations, but those embodiments differ from traditional wired or cabled ICP implants by, for example, enabling long-term monitoring without the cumulative infection risk of exposed hardware (e.g., wires connecting from hardware, through scalp, into brain) and the sonolucent material also allows bedside imaging of a subject's intracranial contents in substantially real-time, among other distinctions. Thus, the combination of ICP monitoring data, obtained via wireless or wired connections, together with valuable ultrasound images and other collected biometric data provides improved implant devices, methods, and other related aspects for synergistically diagnosing, monitoring, and treating civilian, military and veterinary brain injuries. In other words, the ICP and other biometric data obtained using the cranial implant devices disclosed herein provide a long sought after solution for bedside brain imaging combined with telemetric physiological monitoring, in certain aspects.

This application also relates to skull burr holes, burr hole covers, key hole covers, craniotomies or craniectomies with implanted transparent, translucent, sonolucent, acoustically active and acoustically inert materials to create a synthetic window into the skull for diagnostic and/or therapeutic ultrasound, photoacoustic imaging, and/or optical coherence tomography (OCT), among other applications. In certain embodiments, ICP sensors are embedded into, or otherwise associated with, cranial implant housings fabricated from a clear sonolucent or other materials, and then attached to the surrounding cranium (i.e., skull) during a given neurosurgical procedure, using self-containing fixation elements, which may include peripheral extensions fabricated from solid polymethylmethacrylate (PMMA) or other materials with prefabricated holes sized to receive screws or other attachment components. Other attachment features are also provided. In some embodiments, the cranial implant devices described herein are included as part of kits, which optionally include a retractor and/or screws or other attachment components.

In certain embodiments, the cranial implant devices described herein are configured to transmit accurate ICP measurements and/or other biometric data (e.g., brain temperature, tissue CO₂ levels, tissue O₂ levels, and/or the like) in substantially real-time to remote healthcare providers using an associate mobile phone application or other computer readable media implementation. In some embodiments, the sonolucent properties of cranial implant housings also allow direct ultrasound examination at a subject's bedside using the acoustic window, created by the implanted cranial implant device, as effectively an “adult fontanelle”, thus providing continuous data about the underlying brain whenever needed. In addition to enabling real-time, bedside monitoring for purposes of acute and chronic surveillance, the cranial implant devices disclosed herein are also optionally used immediately after implantation into a subject to observe brain structure and to evaluate any symptoms and/or concerns that may arise. In certain aspects, the methods disclosed herein not only produce more effective techniques for detecting acute brain deterioration, but also provide correlations between several sets of biometric data that further inform and guide patient surveillance as well as enhance the understanding of a long-term sequela of various brain pathologies.

Once implanted in subjects, the cranial implant devices disclosed herein may remain in place for indefinite durations. The devices have substantially anatomically-compatible shapes such that they are essentially visually non-detectable to the naked eye upon implantation in subjects. Further, the implantable devices described herein also typically include low-profiles (e.g., to avoid scalp-related complications and high extrusion risk leading to premature explantation). Additional details regarding cranial implant devices, aspects of which are optionally adapted for use with the devices disclosed herein, are found in, for example, International Patent Application No. PCT/US19/39519 and International Patent Publication Nos. WO 2017/039762 and WO 2018/044984, which are each incorporated by reference in their entirety.

Essentially any standardized or customized cranial implant device form is optionally utilized (e.g., circular, elliptical, square, rectangular, triangular, and the like). Additional details regarding customized and/or standardized cranial implants are provided in, for example, U.S. Provisional Patent Application No. 62/155,311, filed on Apr. 30, 2015 and entitled “A Cutting Machine For Resizing Raw Implants During Surgery”, U.S. Provisional Patent Application No. 62/117,782, filed on Feb. 18, 2015 and entitled “Computer-Assisted Cranioplasty”; and International Patent Application No. PCT/US14/67656, filed on Nov. 26, 2014 and entitled “Computer-Assisted Craniomaxillofacial Surgery”, the disclosures of which are each hereby incorporated by reference herein in their entirety.

Surgical access to the intracranial space typically involves a craniectomy or craniotomy. To perform a craniectomy, for example, a series of burr holes or key holes are typically created in the skull. Following surgery, these burr holes may be repaired with a variety of biological materials and/or non-biological materials.

Skull bone generally attenuates, scatters and absorbs ultrasonic waves, thereby limiting transcranial diagnostic and therapeutic ultrasound. Similarly, skull bone is visually opaque, thus limiting the ability to perform transcranial diagnostic photoacoustic imaging or therapeutic light based intervention. By placing materials that transmit acoustic and/or electromagnetic waves in burr holes, as disclosed herein, these limitations can be circumvented.

The size of burr holes previously limited their usefulness as synthetic apertures for transcranial therapeutic ultrasound, diagnostic ultrasound, photoacoustic imaging, optical coherence tomography (OCT), or electromagnetic wave intervention. The cranial implant devices and related aspects disclosed herein modify these synthetic windows, such as by changing the field of view or beam focus to enable the use of these applications previously limited by the size of these standard synthetic window apertures or burr holes.

In certain aspects, the present disclosure provides a skull hole or burr hole ‘plug’ or cranial implant device composed of sonolucent and/or visually translucent biocompatible materials as well a lens or lenses to allow for and enhance the ability to perform transcranial mechanical and/or electromagnetic wave-based diagnostic and therapeutic applications. Applications of post-surgical 2D, 3D, and/or 4D diagnostic ultrasound and photoacoustic imaging include immediate post-operative and long-term diagnostic examination of intracranial pathologies, including, for example, hematomas, brain edema, tumor recurrence, cerebral blood flow, ventricular size, and midline shift. Applications of therapeutic ultrasound and electromagnetic wave intervention, include, for example, lesion ablation, neuromodulation, and blood-brain disruption for targeted delivery of therapeutics, among other techniques.

To further illustrate, in certain exemplary embodiments, the cranial implant devices disclosed herein, which include embedded or otherwise associated pressure sensors (e.g., microsensors or the like), are used in connection with traumatic brain injuries for immediate ICP monitoring and bedside brain imaging for both human and other mammalian subjects. Other neurosurgical cases that can be monitored using these devices are, for example, post-aneurysm surgery, post-brain tumor removal, hydrocephalus surgery and ventricular size monitoring, brain neurodegenerative disease surgery, and post-epilepsy surgery for seizure monitoring and diagnostic aid. In some embodiments, blood flow can be measured throughout the neurological brain anatomy using these devices in cases, such as subarachnoid related cerebral vasospasm, following direct or indirect bypass surgery for Moyamoya disease with single or multiple lenses, and carotid bypass surgery. Optionally, the pressure sensor is used to monitor brain infections, such as meningitis, encephalitis, and brain abscess, among others.

In certain embodiments, the cranial implant devices disclosed herein are implanted in chronic hydrocephalus patients or those having pseurotumor cerebri with slit ventricle syndrome for long-term durations (e.g., weeks, months, or more) to allow valuable, wireless ICP monitoring along with associated brain imaging. In some embodiments, the pressure sensors and bedside brain imaging provided by the cranial implant devices disclosed herein are used for stroke monitoring or brain edema development to guide medical/surgical intervention. Obtaining real-time images via ultrasound through the sonolucent device, in combination with real-time ICP measurements obtained wirelessly, provide the neurological care team with synergistic insight for diagnosis and treatment, as opposed to obtaining only one of those measurements independently and at a different time from the other.

In some embodiments, the cranial implant devices disclosed herein are used as a research tool to aid in monitoring the impact of pharmaceutical treatment and/or surgical treatment for humans or other mammals suffering from brain disease injury. In some of these embodiments, additional vital monitoring technologies/devices are also embedded, or otherwise operably associated with, the cranial implant devices (e.g., monitoring O₂ levels, CO₂ levels, temperature, and/or the like that are wirelessly connected or not). In certain applications, the cranial implant devices disclosed herein are used to permit focused ultrasound to perform treatments on areas of the body other than the brain, such as the liver, lungs, heart, or the like upon being implanted into, for example, a subject's rib cage. In some of these applications, devices implanted in the rib cage are used to provide image-guided surgery for the heart and/or lungs, instead of using CT imaging, as traditionally performed, which exposes the operating surgeon and surgical team to the hazards of regular doses of X-ray radiation. In the case of brain tumors, for example, ultrasound treatments can also aid in the breakdown of the blood-brain barrier (BBB) to facilitate drug delivery for enhanced efficacy.

In certain applications, the cranial implant devices disclosed herein are embedded within a separate customized or non-customized (e.g., standardized) cranial implant (e.g., an alloplastic implant, an autologous implant, etc.). Optionally, these are used for cranioplasty, as a way of providing insight into positively- or negatively-affected intracranial hydrodynamics. In addition, currently, very little is done about the “Syndrome of the Trephined” and how to improve the condition following cranioplasty. The cranial implant devices disclosed herein are valuable to these or other patients, who are in need of skull reconstruction (i.e. cranioplasty) as a result of, for example, suffering from either the Syndrome of the Trephined and/or having pre-operative brain swelling outside the limits of their original cranial boundary (e.g., after sustaining a traumatic brain injury (TBI), stroke, or the like) and need the cranial vault reconstructed with an accompanying reduction in the size of the brain contents back into the original cranial space.

In accordance with embodiments of the present disclosure, a cranial implant device, such as an implantable burr hole plug and/or cover that comprises an acoustic and/or optical lens is provided to create and augment an acoustic, optic or photoacoustic synthetic aperture in the skull. This device typically comprises a single or multiple lens elements assembled within, beneath, and/or over the skull, autologous skull implant or alloplastic skull implant. The lens element may be composed of, for example, electromagnetically translucent, electromagnetically transparent, sonolucent or acoustically active materials. Surrounding and/or between the lens elements may be transparent, sonolucent and/or acoustically inert materials. The lens elements permit and/or enhance transcranial therapeutic ultrasound, diagnostic ultrasound, photoacoustic imaging, electromagnetic wave diagnostic imaging or electromagnetic wave therapeutic intervention. These and other embodiments are described further herein.

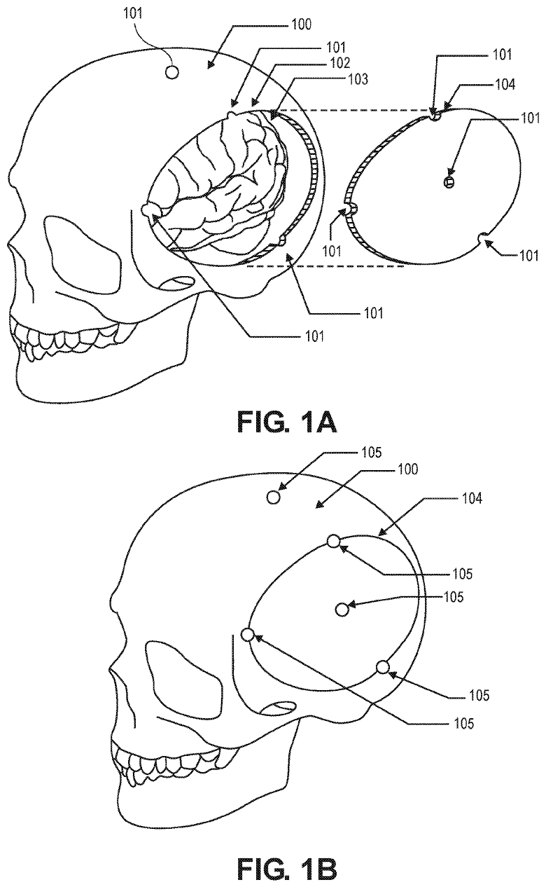

By way of overview, FIGS. 1A and 1B schematically show the insertion of skull bone flap 104, which includes cranial implant device 105 implanted in a burr-hole 101 disposed through skull bone flap 104. During typical cranial surgery, skull bone flap 104 is removed from skull 100 by drilling holes 101, referred to as key holes or burr holes to create craniectomy defect 102 to expose the underlying cranial contents 103. The section of removed bone is typically referred to as a skull bone flap (skull bone flap 104). Additional holes 101 may be placed in skull 100 and/or in a portion of skull 100 and skull bone flap 104. FIG. 1B shows a perspective view of skull 100 with skull bone flap 104 returned to craniectomy defect 102 in skull 100 and cranial implant devices 105 inserted into each burr or key holes 101 in this exemplary embodiment. Depending on the application, not all burr holes 101 are implanted with a cranial implant device 105. In these cases, burr holes 101 created, for example, to remove the skull bone flap 104, may then be repaired by filling them with a variety of biocompatible materials. Following surgical intervention, the craniectomy defect 102 may be filled by returning the skull bone flap 104 and secured or affixed in place using known techniques (e.g., screws, chemical bonding agents, etc.). Alternatively, the skull bone flap 104 can be replaced with an alloplastic or autologous skull implant or flap.

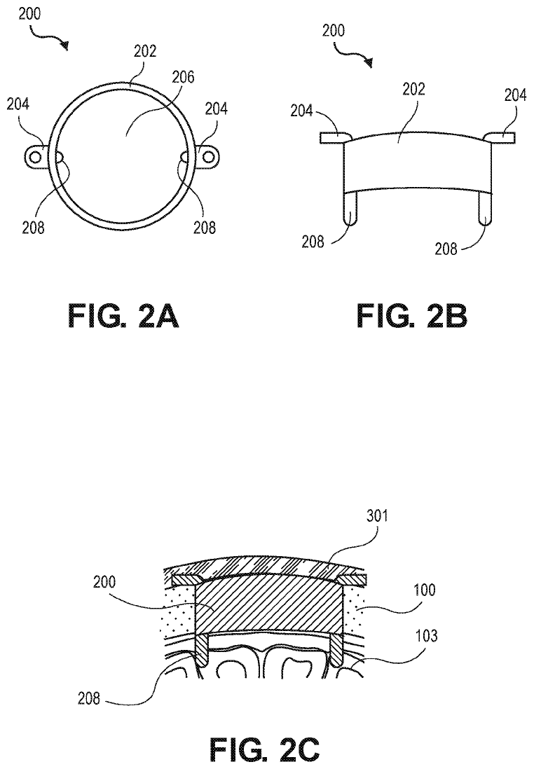

To further illustrate, FIGS. 2A and B schematically show a cranial implant device from top and side views, respectively, according to one exemplary embodiment. As shown, cranial implant device 200 includes cranial implant housing 202 configured for subgaleal scalp implantation within, beneath, and/or over at least one cranial opening of a subject. Cranial implant housing 202 comprises a substantially anatomically-compatible shape and is fabricated from one or more sonolucent materials (and optionally other materials) that permit transmission of mechanical waves (and electromagnetic waves, in certain embodiments) through the sonolucent materials (and other materials when used) when cranial implant device 200 is subgaleally implanted within, beneath, and/or over the cranial opening of the subject. Cranial implant housing 202 also includes attachment features 204 that are configured to receive screws to secure cranial implant device 200 to the subject's skull upon implantation. Lens element 206 is also disposed within cranial implant housing 202. Lens element 206 is optionally fabricated from a variety of materials, as described herein, depending on the intended application. For ultrasound-related applications, for example, lens element 206 is typically also fabricated from one or more sonolucent materials. In certain of these embodiments, cranial implant housing 202 and lens element 206 are fabricated as an integral component (e.g., as an integrally molded part, etc.). Although not shown, in some embodiments, at least one self-sealing access port is disposed through at least a portion a cranial implant device. In these embodiments, the self-sealing access port permits cerebrospinal fluid (CSF) to be aspirated from the subject when the cranial implant device is subgalealy implanted within, beneath, and/or over the cranial opening of the subject.

Cranial implant housing 202 also includes pressure sensors 208 operably connected to cranial implant housing 202. Pressure sensors 208 are configured to sense intracranial pressure (ICP) when cranial implant device 200 is subgaleally implanted within, beneath, and/or over the cranial opening of the subject. Although two pressure sensors are shown, for example, in FIG. 2A, other numbers are also used in the same cranial implant device or in other additionally implanted devices. In some embodiments, for example, on a single pressure sensor is embedded in, or otherwise operably associated with, a given cranial implant device, whereas in other embodiments, more than two pressure sensors (e.g., 3, 4, 5, 6, 7, 8, 9, 10 or more pressure sensors) are embedded in, or otherwise operably associated with, a given cranial implant device. Although not within view in FIGS. 2A-C, cranial implant device 200 also includes one or more controllers (e.g., a first controller) operably connected to pressure sensors 208. Typically, controllers are integrated as part of pressure sensors 208. Controllers are configured to selectively effect pressure sensor 208 to sense the ICP within a cranium of the subject to generate ICP data and to transmit the ICP data to an ICP data receiver when the cranial implant device is subgalealy implanted within, beneath, and/or over the cranial opening of the subject. Cranial implant device 200 includes a power source (not within view) operably connected or connectable at least to the controllers.

To further illustrate, FIG. 2C schematically shows cranial implant device 200 positioned within, beneath, and over a cranial opening of a subject from a sectional side view. As shown, cranial implant device 200 positioned within, beneath, and/or over a cranial opening (e.g., a burr hole) of skull 100 and above intracranial contents 103 and proximal to scalp 301 of the subject. As also shown, pressure sensors 208 extend into intracranial contents 103.

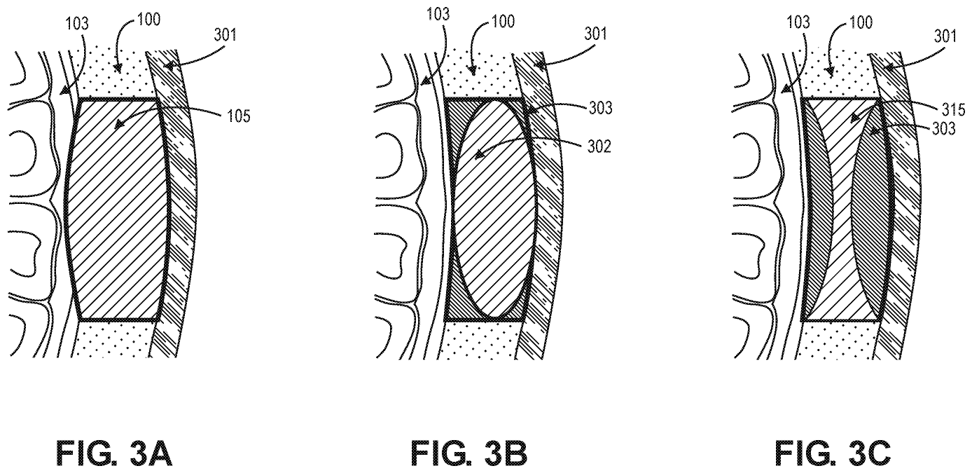

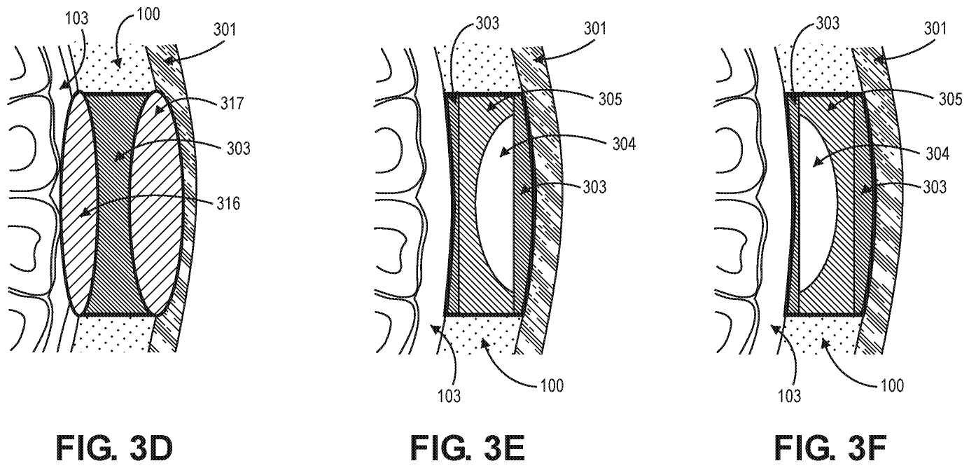

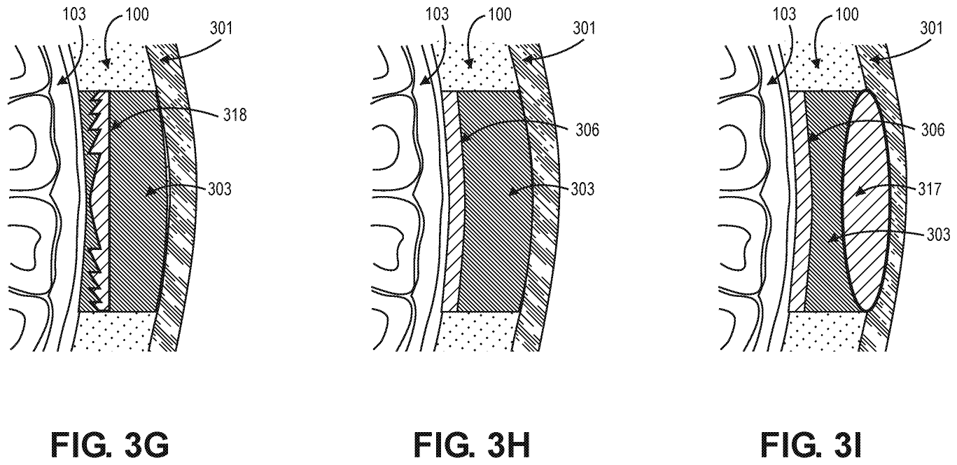

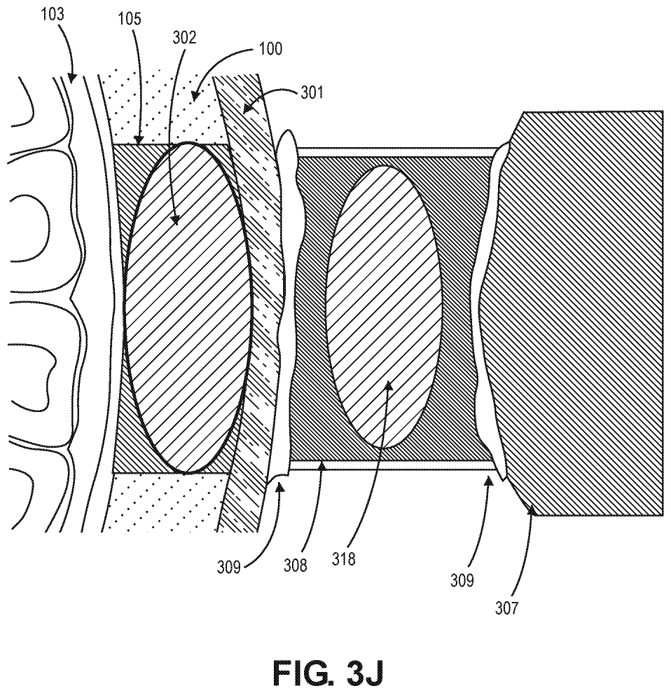

The cranial implant devices disclosed herein optionally include various acoustic, optical, and/or photoacoustic lens elements that include an array of electromagnetically translucent, electromagnetically transparent, sonolucent, and/or acoustically active materials depending on the intended application. Examples of such applications, include transcranial therapeutic ultrasound, transcranial diagnostic ultrasound, photoacoustic imaging, electromagnetic wave diagnostic imaging, and/or electromagnetic wave therapeutic intervention of intracranial matter of a given subject via the acoustic, optical, and/or photoacoustic lens element when the cranial implant device is subgalealy implanted within, beneath, and/or over one or more cranial openings (e.g., burr holes) of the subject. To illustrate, FIGS. 3A-3J schematically show sectional views of various cranial implant devices subgaleally implanted (proximal to scalp 301) within, beneath, and/or over a cranial opening (e.g., a burr hole) of skull 100 and above intracranial contents 103 of a subject according to exemplary embodiments. As described herein, in lieu of, or in addition to, implanting cranial implant devices in burr holes or other cranial openings, cranial implant devices are also optionally implanted in burr holes or other cranial openings disposed through skull bone flaps, autologous skull flaps or implants, and/or alloplastic skull flaps or implants. Cranial openings are typically due to a prior event (e.g., a traumatic brain injury, a concussion, and the like), produced as part of surgery (e.g., craniectomy, cranioplasty, craniotomy, minimally invasive surgery, or the like), or otherwise created specifically to receive the cranial implant devices disclosed herein. Cranial implant devices are typically strategically placed to optimize therapeutic and/or diagnostic applications. Depending on the particular case, a cranial implant device may be implanted as part of an outpatient or inpatient procedure.

In some embodiments, lens elements are curved (e.g., a single or double curved lens, such as a biconcave lens or a biconvex lens) or rectilinear. In certain embodiments, single or multiple lenses (e.g., single or multiple diverging and/or converging lenses) are used in the cranial implant devices disclosed herein. Optionally, a lens element is arranged to create a converging or diverging Fresnel lens. An example of such a lens element configuration is schematically depicted in FIG. 3G. In certain embodiments, lens positions can be adjusted, for example, during and/or after device implantation. Adjustable focus lens elements are optionally automatic or manually adjustable. In some embodiments, adjustable focus lens elements are used in ablative surgical procedures. In some aspects, lens elements are configured to extend into the epidural space or beneath scalp of a given subject. In some embodiments, a cover is created which rests above a given burr hole and acts as an acoustic lens. In some embodiments, the cranial implant devices and/or covers include shapes that customized to match the contours of the skull of a given subject, whereas in the embodiments, the cranial implant devices and/or covers include standardized shapes. In certain embodiments, an adjustable or fixed external lens is used for additional focusing, for example, following device implantation. An example of such an external lens is schematically depicted in FIG. 3J. In certain embodiments, a single lens element or multiple lens elements are integrated within a larger synthetic cranial implant. In some of these embodiments, the larger cranial implant acts as a lens or multiple lenses. The size of these larger cranial implants typically varies depending upon whether the intended subject is a member of the adult or pediatric population.

The cranial implants disclosed herein are fabricated from a wide array of biocompatible materials with varying acoustic and/or optic properties using any known manufacturing technique, including molding processes. These material properties typically allow for transmission of mechanical and/or electromagnetic waves through the materials. Transcranial transmission of these waves permits diagnostic and/or therapeutic applications, including, for example, pathology detection, neuromodulation, and tissue ablation. Modalities which benefit from wave transmission facilitated and enhanced by these devices include, for example, ultrasound, photoacoustic imaging, and optical coherence tomography (OCT), among many others. In addition, as described further herein, these materials may be combined or shaped to alter the transmission of mechanical and/or electromagnetic waves. Effects of altered wave transmission include, for example, increasing the area visible for diagnostic imaging or focusing waves for therapeutic intervention.

The lens elements of the cranial implant devices disclosed herein include a wide variety of properties that can be applied to particular diagnostic and/or therapeutic applications. To illustrate, lens elements are typically fabricated from materials, such as polymethylmethacrylate (PMMA), room-temperature-vulcanizing (RTV) silicone, polydimethylsiloxane (PDMS), epoxy, polyetheretherketone (PEEK), metamaterials, and/or the like. In some embodiments, lens elements are composed of metamaterials with a variety of refractive indices (including negative refractive indices), density, impedance, speed of sound, permittivity, permeability, compressibility, and/or the like. In certain of these embodiments, the use of engineered index materials are employed to achieve imaging beyond the applicable diffraction limit. In some applications, lens elements are composed of acoustic metamaterials and phononic crystals. These materials can simultaneously enhance the field-of-view and the focusing of the incident beam in certain frequencies (i.e., tuned to a certain frequency band). In some embodiments, a combination of various metamaterials and phononic crystals are used to facilitate a broader range of frequency bands.

In some embodiments, lens elements include various three-dimensional patterns/structures of the same material that are used to slow the speed of sound, similar to the effect of sound traveling through other denser materials than air. These patterns/structures form wave-guides that are used to guide waves to trajectories of interest in some embodiments. In certain embodiments, an acoustic lens element is used to accomplish a larger field-of-view by exploiting negative refractive indices, and subwavelength microstructures that are fabricated from non-metamaterials. In certain embodiments, lens elements are fabricated with materials having acoustic properties, which are modified by loading polymers with powders to increase and/or decrease the speed of sound or speed of light within the material.

In some embodiments, a diverging lens element is created using a material through which the speed of sound travels at a lower velocity than in human tissue. In these embodiments, the lens element thickness progressively increases extending radially outward from the center of the lens element. In certain embodiments, a diverging lens element is created using a material through which the speed of sound travels at a greater velocity than in human tissue. In these embodiments, the lens element is thickest at the center and progressively thins extending radially outward from the center of the lens element. In other exemplary embodiments, a flat diverging lens element is created through a combination of at least two different materials. In these embodiments, the speed of sound though these materials transmits at different velocities. One material typically has a greater speed of sound compared to through human soft tissue, while the second material has lower speed of sound compared to through human soft tissue. In some embodiments, a diverging compound concave lens element is used, which includes at least two materials in which the material disposed closest to the scalp of a given subject has a lower speed of sound relative to the material disposed further from the scalp of that subject. In other embodiments, a diverging compound convex lens element is used, which includes at least two materials in which the material disposed closest to the scalp of a given subject has a higher speed of sound relative to the material disposed further from the scalp of that subject. In some embodiments, lens elements with different ratios of focal distance to lens diameter are used. These lens elements are optionally used together or separately to vary the field of view in a given application.

More specifically, FIG. 3A schematically depicts implanted cranial implant device 105 comprising a biconvex lens element from a sectional side view according to one exemplary embodiment. Any of the lens configurations disclosed herein are optionally adapted for use in the cranial implant devices disclosed herein, for example, to facilitate obtaining other biometric data in addition to ICP from the cranial implant devices. As with the other lens elements disclosed herein, implanted cranial implant device 105 allows for the transmission of mechanical and/or electromagnetic waves to and from the intracranial contents 103. FIG. 3B schematically depicts an implanted cranial implant device comprising a biconvex lens element 302 with other material (e.g., sonolucent and/or translucent material 303) disposed on both sides of the lens element from a sectional side view according to one exemplary embodiment. Single or multiple lens elements of different shapes and material properties may be included in the cranial implant device disclosed herein. FIG. 3C schematically depicts an implanted cranial implant device comprising a biconcave lens element 315 with other material (e.g., sonolucent and/or translucent material 303) disposed on both sides of the lens element from a sectional side view according to one exemplary embodiment. FIG. 3D schematically depicts an implanted cranial implant device comprising two biconvex lens elements (316 and 317, respectively) with another layer of material (e.g., sonolucent and/or translucent material 303) disposed between the lens element from a sectional side view according to one exemplary embodiment.

FIG. 3E schematically depicts an implanted cranial implant device comprising a convex lens element 304 with other layers of material (303 and 305, respectively (e.g., sonolucent and/or translucent material)) having different optic and/or acoustic properties disposed on both sides of the lens element from a sectional side view according to one exemplary embodiment. FIG. 3F schematically depicts an implanted cranial implant device comprising a convex lens 304 element with other layers of material (303 and 305, respectively (e.g., sonolucent and/or translucent material)) having different optic and/or acoustic properties disposed on both sides of the lens element from a sectional side view according to one exemplary embodiment. FIG. 3G schematically depicts an implanted cranial implant device comprising a Fresnel lens element 318 with other layers of material (e.g., sonolucent and/or translucent material 303) disposed on both sides of the lens element from a sectional side view according to one exemplary embodiment. FIG. 3H schematically depicts an implanted cranial implant device comprising multiple layers of different materials (e.g., sonolucent and/or translucent material 303, and material which receives optic waves and emits acoustic waves 306) from a sectional side view according to one exemplary embodiment. FIG. 3I schematically depicts an implanted cranial implant device comprising a biconvex lens element 317 with other layers of material (e.g., sonolucent and/or translucent material 303, and material which receives optic waves and emits acoustic waves 306) disposed on one side of the lens element from a sectional side view according to one exemplary embodiment.

FIG. 3J schematically depicts the implanted cranial implant device from FIG. 2B with an external lens element 318 positioned in communication with the implanted cranial implant device and a transmission and/or receiver device from a sectional side view according to one exemplary embodiment. In certain embodiments, this configuration is used to further improve coupling between scalp 300 and ultrasound or photoacoustic transducer 307, a stand-off or gel pad 308, and acoustic gel 309. In some embodiments, external lens element 318 is fabricated integral with stand-off 307 and is optionally adjustable in position. External lens element 318 is typically used to further alter the transmission of acoustic and electromagnetic waves (e.g., by increasing the field of view and/or resolution).

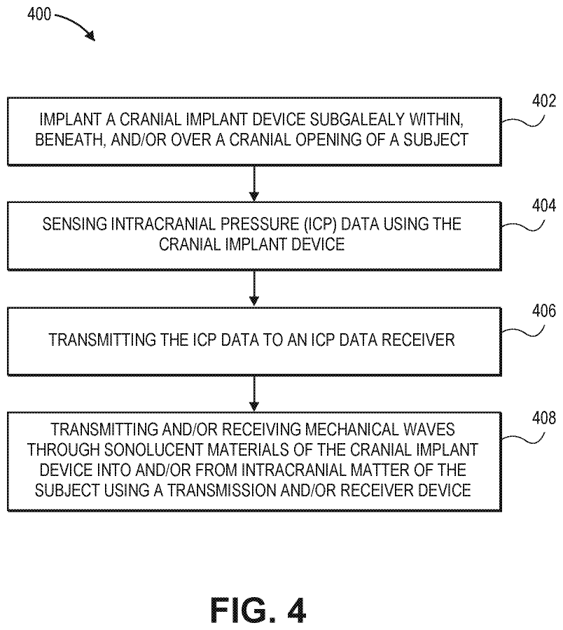

The present disclosure provides various methods of obtaining diagnostic information from, and/or administering therapy to, a subject using the cranial implant devices disclosed herein. To illustrate, FIG. 4 is a flow chart schematically showing such a method according to one exemplary embodiment. As shown, method 400 includes implanting a cranial implant device as described herein subgalealy within, beneath, and/or over a cranial opening (e.g., a burr hole) of the subject in step 402. Typically, step 402 also includes affixing the cranial implant device to a skull of the subject using screws and/or chemical bonding agents. In some embodiments, step 402 also includes positioning a cover over the cranial opening of the subject when the cranial implant device is subgalealy implanted within, beneath, and/or over the cranial opening of the subject, which cover is structured as an acoustic lens. Method 400 also includes sensing the ICP data obtained from the subject using the cranial implant device in step 404, and transmitting the ICP data (via a wireless or a wired connection) to an ICP data receiver from the first controller operably connected to the pressure sensor of the cranial implant device in step 406. Typically, the ICP data to the ICP data receiver is continuously transmitted to the ICP data receiver in substantially real-time. In addition, method 400 also includes transmitting and/or receiving the mechanical waves through the sonolucent materials of the cranial implant device into and/or from intracranial matter of the subject using a transmission and/or receiver device in step 408. Typically, method 400 includes correlating the ICP data with the transmitted and/or received mechanical waves to effect a synergistic diagnosis of, and/or therapeutic administration to, the subject. In certain embodiments, the cranial implant device is retained subgalealy implanted within, beneath, and/or over the cranial opening of the subject for an indefinite duration, for example, in order to continually or periodically monitor ICP and other biometric data from the subject over time.

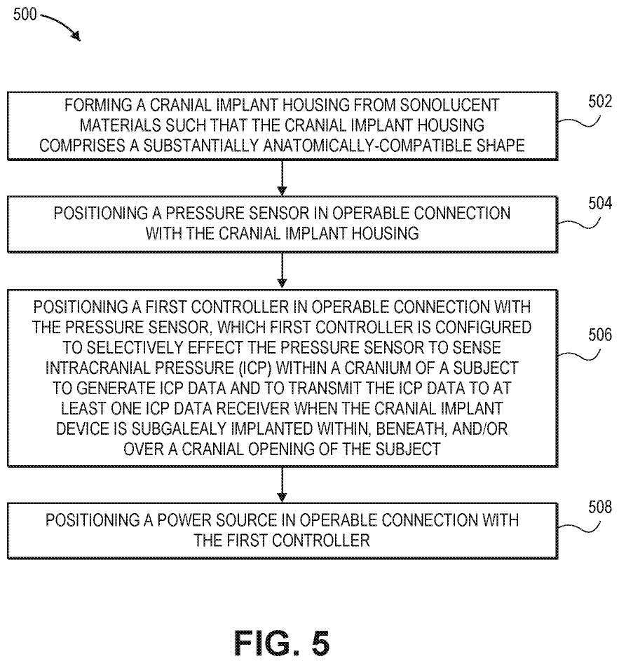

The present disclosure provides various methods of fabricating the cranial implant devices disclosed herein. To illustrate, FIG. 5 is a flow chart schematically showing such a method according to one exemplary embodiment. As shown, method 500 includes forming a cranial implant housing from at least one or more sonolucent materials such that the cranial implant housing comprises a substantially anatomically-compatible shape in step 502. Method 500 also includes positioning at least one pressure sensor (e.g., a pressure microsensor) in operable connection with the cranial implant housing in step 504. The pressure sensor is configured to sense intracranial pressure (ICP) upon implantation in a subject. Method 500 further includes positioning a first controller in operable connection with the pressure sensor in step 506. The first controller is configured to selectively effect the pressure sensor to sense the ICP within a cranium of a subject to generate ICP data and to transmit the ICP data to at least one ICP data receiver when the cranial implant device is subgalealy implanted within, beneath, and/or over a cranial opening of the subject. In certain embodiments, the first controller is manufactured integral with the pressure sensor. In these embodiments, method 500 simply proceeds from step 504 directly to step 508. As shown, step 508 includes positioning at least one power source (e.g., a battery) in operable connection at least with the first controller.

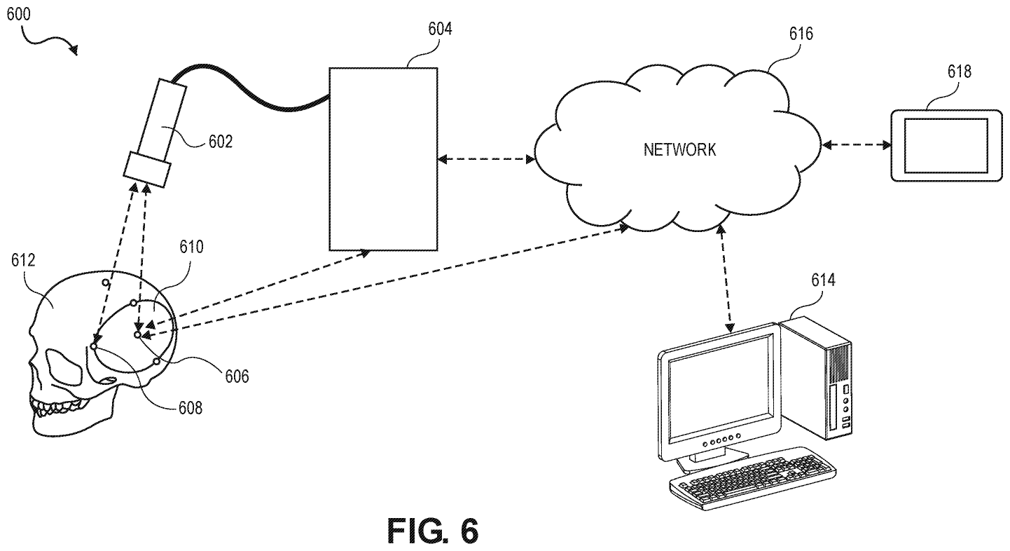

The present disclosure additionally provides a variety of different systems that involve using the cranial implant devices disclosed herein. These systems typically enable, for example, acquiring real-time, non-ionizing, continuous, post-operative monitoring and long-term surveillance of the brain and/or related structures. To illustrate, FIG. 6 schematically shows system 600 according to one exemplary embodiment. As shown, system 600 includes transmission and/or receiver device 602 operably connected to controller 604 (e.g., second controller). Transmission and/or receiver device 602 is configured to transmit and/or receive mechanical and/or electromagnetic waves through cranial implant devices 606 and 608 (as described herein) implanted in burr holes disposed at least partially through skull bone flap 610 of skull 612. In certain embodiments, transmission and/or receiver devices are configured to enable storage, study and modification of received echo signals in a time-domain and/or in a frequency domain. In some embodiments, transmission and/or receiver devices are configured to function as ultrasound devices, photoacoustic devices (including a laser for electromagnetic wave transmission and a receiver for mechanical wave reception), photothermal devices, acousothermal devices, acoustic thermometry devices, and/or optical coherence tomography (OCT) devices. In some embodiments, system 600 further includes acoustic microscopy functionality. The first controllers of cranial implant devices 606 and 608 are also configured to wirelessly transmit ICP data sensed by the pressure sensors to controller 604, and/or to computer 614 and/or mobile device 618 via network 616. In some of these embodiments, the first controllers of cranial implant devices 606 and 608 are operably connected to a cabled or wireless ICP monitor (e.g., having an external antenna), which is operably connected with, or manufactured integral with, controller 604.

Controller 604 comprises, or is capable of accessing, computer readable media comprising non-transitory computer-executable instructions which, when executed by an electronic processor, cause transmission and/or receiver device 602 to transmit and/or receive mechanical and/or electromagnetic waves through the acoustic, optical, and/or photoacoustic lens element (e.g., comprising sonolucent and optionally other materials) of cranial implant devices 606 and 608 when transmission and/or receiver device 602 is positioned in communication with cranial implant devices 606 and 608. As also shown, controller 604 is wirelessly connected with computer 614 via network 616 (as indicated by, for example, the dashed-lines between controller 604 and network 616, and between network 616 and computer 614). Optionally, controller 604 and computer 614 are operably connected to network 616 via wired connections. In other embodiments, controller 604 and computer 614 are operably connected to one another directly (i.e., not via network 616) via a wired or wireless connection, whereas in other exemplary embodiments, controller 604 comprises computer 614.

While not limited to any particular embodiment, computer 614 may be a desktop computer, notebook computer, smart phone, tablet, a virtual reality device, a mixed reality device and network 616 may be a cloud server or another format. In certain embodiments, computer 614 displays data associated with mechanical and/or electromagnetic waves sent from, and/or received by, transmission and/or receiver device 602 during the course (e.g., in substantially real-time) of a given diagnostic and/or therapeutic application.

In some embodiments, the systems disclosed herein include an ultrasound transducer that is modified to send and receive ultrasound waves transmitted through a lens element of an implanted cranial implant device. Transducers may be concave, convex, flat or a combination of geometries. Modifications may include an application specific imaging sequence or synthetic aperture imaging technique embodied in non-transitory computer readable media. Transducer parameters that are optionally varied, include number of elements, center frequency, speed of sound, wave length, array pitch, sampling frequency, emission pulse, and the like. In some embodiments, ultrasound systems include non-transitory computer readable media to reassemble, normalize, and otherwise process images transmitted through lens elements of implanted cranial implant devices (e.g., in substantially time).

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, cranial implant devices, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. An implant device, comprising:
at least one implant housing configured for implantation within, beneath, and/or over at least one cranial opening or bodily opening of a subject, which implant housing comprises a substantially anatomically-compatible shape and is fabricated from one or more sonolucent materials that permit transmission of one or more mechanical waves through the sonolucent materials when the implant device is implanted within, beneath, and/or over the cranial opening or bodily opening of the subject;
at least one pressure sensor operably connected to the implant housing, which pressure sensor is configured to sense intracranial pressure (ICP) or sense intrabodily pressure (IBP);
at least a first controller operably connected to the pressure sensor, in which the first controller is configured to selectively effect the pressure sensor to sense the ICP or IBP within a subject to generate ICP or IBP data and to transmit the ICP or IBP data to at least one ICP or IBP data receiver when the implant device is implanted within, beneath, and/or over the cranial opening or bodily opening of the subject; and,
at least one power source operably connected or connectable at least to the first controller.

2-4. (canceled)

5. The implant device of claim 1, wherein the pressure sensor, the first controller, and/or the power source are at least partially embedded in the implant housing.

6-10. (canceled)

11. The implant device of claim 1, wherein the mechanical waves comprise ultrasound waves.

12. The implant device of claim 1, wherein the pressure sensor comprises a micro sensor.

13-16. (canceled)

16. The implant device of claim 1, wherein the first controller is operably connected to the ICP or IBP data receiver via at least one connection and wherein the first controller is further configured to transmit the ICP or IBP data to the ICP or IBP data receiver via the connection when the implant device is implanted within, beneath, and/or over the cranial opening or bodily opening of the subject.

17-21. (canceled)

22. The implant device of claim 5, wherein the ICP data receiver comprises a mobile device selected from the group consisting of: a telephone, a tablet computer, and a notebook computer.

23. The implant device of claim 1, wherein the sonolucent materials form at least one lens element.

24-27. (canceled)

28. The implant device of claim 23, wherein the lens element comprises one or more wave-guides.

29. The implant device of claim 23, wherein the lens element comprises one or more acoustic metamaterials and/or one or more phononic crystals.

30. The implant device of claim 23, wherein the lens element comprises at least one metamaterial having a negative refractive index and at least one other material having a subwavelength microstructure.

31-32. (canceled)

33. The implant device of claim 23, wherein the lens element comprises at least one material that is modified to increase or decrease a speed of sound transmitted through the material.

34. The implant device of claim 23, wherein the lens element comprises:
(a) at least one substantially flat diverging lens comprising at least two different materials, wherein at least a first material transmits sound at a higher speed than a tissue of the subject, and wherein at least a second material transmits sound at a lower speed than the tissue of the subject; or
(b) at least one diverging compound concave lens comprising at least two different materials, wherein at least a first material transmits sound at a higher speed than at least a second material, and wherein the second material is positioned closer to a scalp of the subject than the first material when the implant device is subgalealy implanted within, beneath, and/or over the at least one cranial opening of the subject; or
(c) at least one diverging compound convex lens comprising at least two different materials, wherein at least a first material transmits sound at a lower speed than at least a second material, and wherein the second material is positioned closer to a scalp of the subject than the first material when the implant device is subgalealy implanted within, beneath, and/or over the at least one cranial opening of the subject; or
(d) at least two lenses, wherein at least a first lens comprises a different ratio of focal distance to lens diameter than at least a second lens; or
(e) at least one diverging lens that transmits sound at a lower speed than a tissue of the subject.

35-39. (canceled)

40. The implant device of claim 23, wherein the lens element comprises at least one diverging lens that transmits sound at a lower speed or a higher speed than a tissue of the subject.

41-43. (canceled)

44. A cranial implant device, comprising:
at least one acoustic, optical, and/or photoacoustic lens element comprising one or more electromagnetically translucent, electromagnetically transparent, sonolucent, and/or acoustically active materials; and
at least one biometric data sensor operably connected to the acoustic, optical, and/or photoacoustic lens element, wherein the biometric data sensor is configured to sense biometric data from a cranium of a subject and to transmit the biometric data to at least one ICP data receiver;
wherein the cranial implant device is structured for subgaleal scalp implantation within, beneath, and/or over at least one cranial opening of the subject;
wherein the cranial implant device comprises a substantially anatomically-compatible shape; and,
wherein the cranial implant device further permits transcranial therapeutic ultrasound, transcranial diagnostic ultrasound, photoacoustic imaging, electromagnetic wave diagnostic imaging, and/or electromagnetic wave therapeutic intervention of intracranial matter of the subject via the acoustic, optical, and/or photoacoustic lens element when the cranial implant device is subgalealy implanted within, beneath, and/or over the at least one cranial opening of the subject.

45-56. (canceled)

57. A system, comprising:
at least one cranial implant device, comprising:
at least one cranial implant housing configured for subgaleal scalp implantation within, beneath, and/or over at least one cranial opening of a subject, which cranial implant housing comprises a substantially anatomically-compatible shape and is fabricated from one or more sonolucent materials that permit transmission of one or more mechanical waves through the sonolucent materials when the cranial implant device is subgalealy implanted within, beneath, and/or over the cranial opening of the subject;
at least one pressure sensor operably connected to the cranial implant housing, which pressure sensor is configured to sense intracranial pressure (ICP);
at least a first controller operably connected to the pressure sensor, which first controller is configured to selectively effect the pressure sensor to the ICP within a cranium of the subject to generate ICP data and to transmit the ICP data to at least one ICP data receiver when the cranial implant device is subgalealy implanted within, beneath, and/or over the cranial opening of the subject; and
at least one power source operably connected or connectable at least to the first controller;
at least one transmission and/or receiver device configured to transmit and/or receive the mechanical waves through the sonolucent materials when the cranial implant device is subgalealy implanted within, beneath, and/or over the cranial opening of the subject; and,
at least a second controller operably connected to the transmission and/or receiver device, which second controller comprises, or is capable of accessing, computer readable media comprising non-transitory computer-executable instructions which, when executed by at least one electronic processor cause the transmission and/or receiver device to transmit and/or receive the mechanical waves through the sonolucent materials of the cranial implant device when the cranial implant device is subgalealy implanted within, beneath, and/or over the cranial opening of the subject and when the transmission and/or receiver device is positioned in communication with the cranial implant device.

58-98. (canceled)
